(19) United States
(12) Patent Application Publication
(10) Pub. No.: US 2003/0157088 A1
(43) Pub. Date: Aug. 21, 2003

(54) Compositions containing enzymes stabilized with inhibitors having certain binding properties and methods for using such compositions in personal care

(75) Inventors: Russell Phillip Elliott, Egham (GB); Charles Winston Saunders, Fairfield, OH (US); David John Weisgerber, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Proctor & Gamble Company, Cincinnati, OH

(21) Appl. No.: 10/293,100

(22) Filed: Nov. 12, 2002

Related U.S. Application Data
(60) Provisional application No. 60/338,149, filed on Nov. 13, 2001.

(51) Int. Cl. A61K 38/48

(52) U.S. Cl. 424/94.64; 424/94.63

(57) ABSTRACT

Leave-on personal care compositions comprising a stabilized enzyme, at least 30% water, less than 5% of an earth alkali metal salt, and an enzyme inhibitor having an inhibition constant (Kₗ) from 10 nanomolar (nM) to 25,000 nM. Also, a method of providing a skin care benefits, e.g. skin feel and/or skin appearance, preferably skin moisturization, skin softness, and/or skin smoothness, comprising topically applying a safe and effective amount of the inventive compositions described herein to skin in need of such a skin care benefit. Further, a method of providing a consumer with a leave-on enzyme-containing personal care product, wherein the enzyme is inhibited and inactive on shelf, but is active on skin without the need for a separate re-activation step, preferably wherein the leave-on enzyme-containing personal care product comprises a one of the inventive compositions described herein.

### CROSS REFERENCE TO RELATED APPLICATION

This application claims the benefit of U.S. Provisional Application No. 60/338,149, filed Nov. 13, 2001.

### BACKGROUND OF THE INVENTION

This invention relates to leave-on personal care compositions comprising a stabilized enzyme and an enzyme inhibitor having an inhibition constant (Kₗ) having a value within a range described herein, and use of such compositions for personal care to provide improved skin feel and appearance, particularly skin moisturization, skin softness and skin smoothness. The skin of a mammal, preferably a human, but including horses, dogs, cats, and other lower animals, is made up of several layers of cells that coat and protect the keratin and collagen fibrous proteins that form the skeleton of its structure, the outermost of these layers being commonly referred to as the stratum corneum. Normally, the dead cells contained in this layer are desquamated, but many environmental factors and washing with cleansers may interfere with this process, leading to a build-up of dead cells, resulting in dry skin. For example, anionic surfactants and organic solvents typically penetrate the stratum corneum, and by delipidization (i.e. removal of the lipids from the stratum corneum), destroy its integrity. This destruction of the skin surface topography may lead to a rough feel and may eventually permit the surfactant, solvent, or agents carried by them, to interact with the keratin, causing irritation. Dry, itchy, or flaky skin may result from failure to maintain a proper water gradient across the stratum corneum. Most of the water needed to maintain this gradient comes from inside the body. If the humidity is too low, such as in a cold and/or arid climate, insufficient water remains in the outermost portions of stratum corneum to properly plasticize the tissue, and the skin begins to dry up, flake, and/or become itchy.

The use of enzymes in personal care compositions to provide a skin care benefits per se is known. It is believed that enzymes, particularly proteases, function primarily by providing a desquamatory action to the skin on which such compositions are applied. It is believed that the enzymes may remove damaged (e.g. dry or flaky) skin cells on the surface of the skin, thereby reducing the rough feel associated therewith, and allowing skin care actives to reach more live skin cells, thereby enhancing their activity. As the enzymes diminish the effect of prior damage to the skin, they may give the skin a fresher, more youthful appearance and feel. Thus, it is desirable to improve skin condition by topically applying enzymes, preferably proteases, optionally with other skin active agents in a safe (to the consumer) and stable (on shelf) form. It is also desirable to derive improved performance of skin active agents by combining them with enzymes, preferably proteases.

However, there are problems associated with the inclusion of enzymes in personal care compositions. The overall stability of an enzyme is comprised of its thermal stability, pH stability, oxidative stability, and conformational stability. A problem with stabilizing enzymes in product is that enzymes, particularly proteases, are unstable in water, and rapidly undergo auto-digestion. Several solutions to this problem have been suggested.

One approach would be to inhibit the activity of the enzyme in product by adding enzyme inhibitors (e.g. direct inhibitors that block the enzyme's active site or indirect inhibitors which produce a reaction product that drives the auto-digestion reaction's overall equilibrium back to the left (away from auto-digestion)) to reduce the rate at which the auto-digestion reaction takes place. Typically, the more the rate is reduced, the longer the enzyme is stable on shelf. To achieve a commercially viable shelf-life for a personal care product, though, the rate must be slowed nearly to a stop, rendering the enzyme inactive. Other approaches would be to encapsulate the enzyme prior to inclusion within the personal care product (see GB 1,255,284 and JP 10-251,122); or to buffer the composition such that the enzyme remains inactive until use (see WO 97/47,238); or to formulate an anhydrous composition; or to dramatically reduce water activity in the composition by adding very high levels of polyhydric alcohols (see JP 1,283,213 and JP 3,294,211 and EP 755,673 and EP 759,293); or to formulate various emulsions (see EP 779,071); or to separately package a first compartment containing a stabilized enzyme and a second compartment containing an activator, and mix during use (see WO 97/27,841). However, what complicates these suggested solutions is that once applied to the skin of a consumer, the enzyme must be fully active in order to provide skin care benefits. This requires a separate re-activation step, e.g. to dilute an enzyme-containing product, in which the enzyme has been de-activated with inhibitors during shelf storage, with water to re-activate the enzyme on skin or to rupture the emulsion or encapsulation, or to mix two precursor products into one on the skin, etc. It has also been proposed to use ectoin or derivatives thereof to inhibit enzymatic activity (see DE 198 34 816 and WO 01/54,446), however, the use of such osmo-protectants may not provide sufficiently improved stability and activity benefits desired in a personal care product. Further, use of inhibitors to stabilize enzyme-containing products has heretofore been limited to formulation as a rinse-off product, because the subsequent step of dilution has been needed to re-activate the enzyme.

While it is known to use some inhibitors to de-activate enzyme in product, it has not been previously known to use inhibitors having certain binding properties to stabilize enzymes in aqueous leave-on personal care products. Applicants have surprisingly found that inhibitors having an inhibition constant (Kᵢ) within a certain range may provide for enzyme-containing leave-on compositions wherein the enzyme is stable (de-activated) on shelf, but is made active on skin without the need for a separate re-activation step. Without being bound by theory, it is believed that inhibitors having a Kᵢ within this range are able to bind to the enzyme tightly enough to stabilize the enzyme on shelf, but not so tightly that when the leave-on composition is applied to skin a separate re-activation step is needed. During usage, sufficient inhibitor “comes off” the enzyme to allow the enzyme to activate. Since the leave-on product is applied as a layer to the skin, the inhibitor concentration is significantly exceeded by the available substrate (i.e. proteins of skin), and the probability of the enzyme-inhibitor complex forming is significantly reduced. The inventive compositions will not require a further re-activation step, and they may be stored for a commercially viable period. Such compositions may provide significant improvements in stability of enzymes while maintaining their efficacy. Further, such compositions may be useful to improve skin condition and improve performance of skin active agents.

### SUMMARY OF THE INVENTION

The present invention relates to leave-on personal care compositions comprising (a) a stabilized enzyme, (b) at least 30% water, (c) less than 5% of an earth alkali metal salt, and (d) an enzyme inhibitor having an inhibition constant (Kᵢ) from 10 nanomolar (nM) to 25,000 nM. The invention also relates to a method of providing a skin care benefits, e.g. skin feel and/or skin appearance, preferably skin moisturization, skin softness, and/or skin smoothness, comprising topically applying a safe and effective amount of the inventive compositions described herein to skin in need of such a skin care benefit. The invention further relates to a method of providing a consumer with a leave-on enzyme-containing personal care product, wherein the enzyme is inhibited and inactive on shelf, but is active on skin without the need for a separate re-activation step, preferably wherein the leave-on enzyme-containing personal care product comprises one of the inventive compositions described herein.

### DETAILED DESCRIPTION

The compositions, when applied topically to the skin of a mammal, may provide improved skin feel and appearance, particularly skin moisturization, skin softness and skin smoothness. The compositions of the present invention can comprise, consist of, or consist essentially of the essential elements and limitations of the invention described herein, as well as any of the additional or optional ingredients, components or limitations described herein. The components of the compositions of the present invention, including those that may optionally be added, as well as methods for preparation, and methods for use thereof, and several exemplary embodiments are described in detail below. Except as otherwise noted, amounts represent approximate weight percent of the actual amount of the ingredient, and do not include solvents, fillers or other materials which may be combined with the ingredient in commercially available products, and the amounts include the composition in the form of intended use.

Except as otherwise noted, all amounts including parts, percentages and proportions are understood to be modified by the word “about,” and amounts are not intended to indicate significant digits. Except as otherwise noted, the articles “a,” “an,” and “the” mean “one or more.” All publications cited herein are hereby incorporated by reference in their entirety.

The term “safe and effective amount” as used herein, means an amount of an active ingredient high enough to modify the condition to be treated or to deliver the desired skin care benefit, but low enough to avoid serious side effects, at a reasonable benefit to risk ratio within the scope of sound medical judgment. What is a safe and effective amount of the active ingredient will vary with the specific active, the ability of the active to penetrate through the skin or hair, the age, health condition, and skin or hair condition of the user, and other like factors.

As used herein, “pharmaceutically-acceptable” means that drugs, medications or inert ingredients which the term describes are suitable for use in humans and lower animals without undue toxicity, incompatibility, instability, irritation, allergic response, and the like. As used herein, “cosmetically acceptable” means that ingredients which the term describes are suitable for use in contact with the skin or hair of humans and lower animals without undue toxicity, incompatibility, instability, irritation, allergic response and the like.

The term “enzyme” as used herein means the enzyme, wild-type or variant, either per se, or chemically modified by the conjugation of polymer moieties. The term “protease enzyme” as used herein refers to any enzyme whose substrate is a protein. As used herein, the term “wild-type” refers to an enzyme produced by unmutated hosts. As used herein, the term “variant”, means an enzyme having an amino acid sequence which differs from that of the wild-type enzyme due to the genetic mutation of the host producing that enzyme.

The term “leave-on” as used herein means that the compositions being described as such are intended for use without an immediate rinsing step, i.e. that after topically applying the composition to the skin, the composition is typically left on the skin for a period of at least 2, preferably 15 minutes, more preferably at least an hour, more preferably several hours, and possibly overnight.

Abbreviations used herein for amino acids are set forth in the following table. Substitutions of amino acids are denoted by indicating the original amino acid, followed by the amino acid position, followed by the amino acid that is being substituted in, e.g. “N76D” indicates that asparagine at position 76 is replaced by aspartic acid. Combinations of substitutions are shown with dashes “-” between them, e.g. “N76D-I122A-Y217L” indicates what substitutions have taken place at positions 76, 122, and217.

| | | | | | |
|---|---|---|---|---|---|
| A | Ala | Alanine | M | Met | Methionine |
| B | Asx | Asparagine or Aspartic | N | Asn | Asparagine |
| | | Acid | | | |
| C | Cys | Cysteine | P | Pro | Proline |
| D | Asp | Aspartic Acid | Q | Gln | Glutamine |
| E | Glu | Glutamic Acid | R | Arg | Arginine |
| F | Phe | Phenylalanine | S | Ser | Serine |
| G | Gly | Glycine | T | Thr | Threonine |
| H | His | Histidine | V | Val | Valine |
| I | Ile | Isoleucine | W | Trp | Tryptophan |
| K | Lys | Lysine | Y | Tyr | Tyrosine |
| L | Leu | Leucine | Z | Glx | Glutamine or Glutamic |
| | | | | | Acid |

### I. Components

The compositions of the present invention comprise an enzyme, water, and certain inhibitors having a range of Kᵢ described herein, and a minimal amount, if any, of earth alkali metal salts. The ingredients comprising the compositions herein, as well as other optional components, are described in detail as follows. As is known in the art, many cosmetic ingredients have multiple functions in formulations and therefore may be included in several functional groupings. Accordingly, it should be understood that although the active ingredients useful herein are categorized by their therapeutic benefit or their postulated mode of action, some such ingredients can in some instances provide more than one cosmetic and/or therapeutic benefit or operate via more than one mode of action. Therefore, classifications herein are made for the sake of convenience and are not intended to limit the active ingredient to that particular application or applications listed. Also, where not stated otherwise, cosmetically and pharmaceutically acceptable salts of these active ingredients are useful herein.

A. Enzyme

The compositions of the present invention comprise as an essential component, an enzyme in an amount that is sufficiently effective to exfoliate the epidermis, e.g. facilitate the removal of dry skin flakes and/or to enhance the activity of skin active agents. Typically a safe and effective amount will range from 0.0001% to 1%, preferably from 0.0005% to 0.5%, more preferably from 0.001% to 0.1%. Suitable enzymes for use herein include but are not limited to proteases and lipases (e.g. those lipases described in U.S. Pat No. 6,284,246B1 (Procter & Gamble)).

Proteases are classified under the Enzyme Classification number E.C. 3.4 (Carboxylic Ester Hydrolases) in accordance with the Recommendations (1992) of the International Union of Biochemistry and Molecular Biology (IUBMB). Proteases suitable for use herein are also described in PCT publications WO 95/30010, WO 95/30011, and WO 95/29979, all three published Nov. 9, 1995 (Procter & Gamble) and in U.S. Pat. No. 6,284,246B1 ((Procter & Gamble) especially those named as Proteases “A” to “F” in said document). Preferred proteases for use herein include, but are not limited to subtilisin, chymotrypsin and elastase protease enzymes and variants thereof, more preferably subtilisins, those proteases having homology to subtilisins (“subtilisin-like”) and variants of either. Subtilisin enzymes are naturally produced by Bacillus alcalophilus, Bacillus amyloliquefaciens, Bacillus anlylosaccharicus, Bacillus licheniformis, Bacillus lentus and Bacillus subtilis microorganisms. The amino acid sequences of several subtilisins are known, and are set forth, for example, in WO 89/06279, published on Jul. 13, 1989 (Novo Nordisk). Suitable subtilisins for user herein include but are not limited to subtilisin BPN′, subtilisin Carlsberg, subtilisin DY, subtilisin 147, subtilisin 168, subtilisin 309, and subtilisin amylosaccaritus, preferably subtilisin BPN′. Also suitable are papain, bromelain, thermitase, and aqualysin.

Suitable proteases for use herein also include variants, preferably variants of subtilisins and their homologues, having an amino acid sequence modified by addition, substitution, or deletion at one or more of the following positions: 5, 19, 22, 32, 33, 36, 41, 50, 64, 68, 71, 75, 76, 77, 78, 79, 80, 81, 87, 89, 104, 109, 115, 116, 117, 119, 120, 122, 131, 136, 151, 153, 155, 156, 166, 169, 170, 171, 172, 173, 174, 176, 181, 189, 193, 195, 196, 206, 208, 209, 211, 214, 217, 218, 219, 222, 235, 251, and 271.

Preferred substitutions include, but are not limited to P5S, R19G, Q19G, T22C, M50F, V68M, V68C, T71E, T71D, N76D, N77D, S78D, I79A, I79E, S87C, E89S, N109I115A, A116V, N117A, M119A, H120D, I122A, G131D, S153A, G169A, K170D, R170Y, Y171Q, P172D, P172E, S173D, N181D, V193M, G195Q, G195D, Q206D, Q206V, Q206C, Y209L, L211D, Y217K, Y217L, N218D, N218S, G219C, M222C, M222A, K235L, K251E, and Q271 E. Preferred combinations of substitutions include, but are not limited to: (a) N76D-I122A-Y217L, (b) 179A-I122A-Y217L, (c) N76D-179A-1122A-Y217L, and (d) any of combinations a to c further combined with one or more substitutions selected from P40Q, D41A, Q206L, N218S, and H238Y.

Preferred deletions include, but are not limited to elimination of the amino acids at positions 75 to 83. Preferred substitutions to be further combined with this deletion include substitutions at one or more of positions 9, 31, 126, 156, 166, 169, 188, 212, 217, 222, and 254, more preferably S9A, I31L, E156S, G166S, G169A, S188P, N212G, K217L, L126I, M222Q, T254A. Another preferred deletion includes the elimination of the amino acids at positions 75 to 83 and deletion of one or more amino acids at positions 1 to 22, preferably all of 1 to 17. Preferred substitutions to be further combined with these deletions include substitutions at one or more of positions 2, 3, 5, 9, 31, 43, 50, 73, 126, 156, 166, 169, 188, 206, 212, 217, 218, 221, 222, 254 and 271, more preferably Q2K, S3C, P5A, S9A, I31L, K43N, M50F, A73L, L126I, E156S, G166S, G169A, S188P, Q206C, N212G, K217L, N218S, S221C, M222Q, T254A and Y271K.

Specific non-limiting examples of suitable lipases are LIPOLASE (RTM), LIPOLASE ULTRA (RTM), LIPOZYME (RTM), PALATASE (RTM), NOVOZYM 435 (RTM), LECITASE (RTM), all available from Novo Nordisk (Denmark), and LUMAFAST (RTM), LIPOMAX (RTM), both available from Genencor (San Franscisco). Specific non-limiting examples of suitable proteases are ALCALASE (RTM), ESPERASE (RTM), SAVINASE (RTM), all available from Novo Nordisk (Denmark), and MAXATASE (RTM), MAXACAL (RTM), MAXAPEM 15 (RTM), all available from Gist-Brocades (Netherlands), and subtilisin BPN′, which are commercially available.

B. Water

The compositions of the present invention comprise as an essential component, water in an amount that is sufficiently effective to enhance the activity of the enzyme and to solubilize the other essential ingredients of the inventive compositions. Typically a safe and effective amount will be at least 30%, preferably at least 40%, more preferably at least 50%.

The “water activity a_{w}” of a medium containing water is the ratio of the water vapour pressure of the product “P_{H20} product” to the vapour pressure of pure water “P_{H2O} pure” at the same temperature. It can also be expressed as the ratio of the number of molecules of water “N_{H2O}” to the total number of molecules “N_{H2O}+N_{dissolved substances}”, which takes account of the molecules of dissolved substances “N_{dissolved substances}”. It is given by the following formulae:

$${a}_{w} = \frac{{}_{}^{P}{H}{}_{2}^{} {O}^{product}}{{}_{}^{P}{H}{}_{2}^{} O} = \frac{{}_{}^{N}{H}{}_{2}^{} O}{{}_{}^{N}{H}{}_{2}^{} O {+}^{N} dissolved substances} .$$

Various methods can be used for measuring the water activity. The most common is the manometric method, by which the vapour pressure is measured directly. Typically, compositions of the present invention will have a water activity greater than 0.65, preferably greater than 0.75, more preferably greater than 0.85. Typically, compositions of the present invention will have a low (e.g. less than 10 atmospheres) osmotic pressure.

C. Earth Alkali Metal Salt

The inventive compositions comprise a minimal, if any, amount of an earth alkali metal salt sufficiently effective to enhance the stability of the enzyme. Typically a safe and effective amount will bed less than 5%, preferably less than 0.1%, more preferably less than 0.05%, still more preferably less than 0.02%, still yet more preferably less than 0.015%. While not being bound by theory, it is believed that the earth alkali metal salt may insert into the calcium binding site of the enzyme, thereby conformationally stabilizing it, however in excess of 5%, the earth alkali metal salt may de-stabilize the enzyme by increasing auto-digestion due to osmotic effects. Such earth alkali metals include magnesium, calcium, and strontium.

D. Enzyme Inhibitors

The inventive compositions comprise as an essential component an enzyme inhibitor stabilizer in an amount that is sufficiently effective to reduce the rate of auto-digestion of the enzyme, thereby enhancing the stability of the enzyme. Typically a safe and effective amount will range from 0.00005% to 5%, preferably from 0.005% to 2%, more preferably from 0.025% to 1%. Kᵢ values reported herein are approximate values and may be measured, e.g. using the method set forth in “Kinetcs of Subtilisin and Thiolsubtilisin“, Philipp, M. and Bender, M. L., Molecular & Cellular Biochemistry, vol. 51, pages 5-32 (1983). Another suitable method is set forth in “Protease Stabilization by Carboxylic Acid Salts: Relative Efficencies and Mechanisms,” Crossin, Michael C., J.A.O.C.S. vol. 66, no. 7, pages 1010-1014 (July 1989). Inhibitors suitable for use herein will have an inhibition constant such that the inhibitor exhibits the ability to bind to the enzyme in product, but then have a sufficient “off-rate” upon application to skin that the enzyme will be active on skin without the need for a separate re-activation step. Inhibitors having too low a Kᵢ will tend to bind too tightly to the enzyme and not allow the enzyme to be active on skin, and inhibitors having too high a Kᵢ will tend not to bind sufficiently tightly enough to stabilize the enzyme on shelf As used herein “off-rate” is the velocity at which the enzyme-inhibitor complex breaks down to release uninhibited enzyme. The inhibition constant (Kᵢ) is defined as the concentration of inhibitor required to inhibit an enzyme's activity by 50%, and may be represented as Kᵢ=[E]*[I]/[EI], wherein E is enzyme, I is inhibitor, and EI is enzyme-inhibitor complex. Inhibitors suitable for use herein will typically have a Kᵢ from 10 nM to 25,000 nM, preferably from 50 nM to 5,000 nM, more preferably from 100 nM to 1,000 nM. Preferably, the molar ratio of enzyme to inhibitor is from 2:1 to 1:20, more preferably from 1:1 to 1:15.

Suitable inhibitors include but are not limited to aryl boronic acid derivatives according to the following formulae:

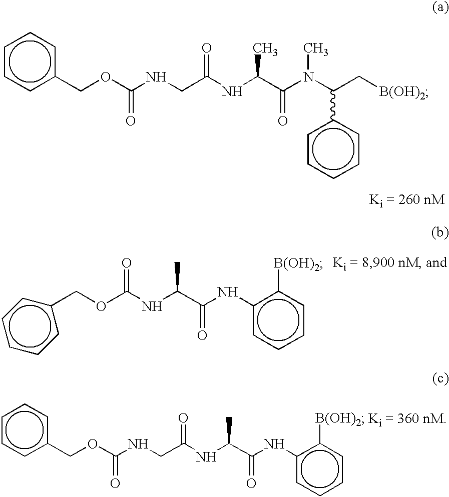

Suitable inhibitors for use herein also include an enzyme inhibitor conjugate having the formula:

Poly]-(L)_{z}-[R—CHO

wherein [Poly] is a water-soluble, non-peptidic polymer component, L is an optionally present linking group, y has the value of at least 1; z has the value 0 or 1, and —RCHO is a substrate (capable of interacting with one or more enzymes to reversibly inhibit the enzyme) having the formula:

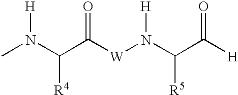

wherein R⁴ and R⁵ are each independently selected from the group consisting of substituted or unsubstituted: phenyl, benzyl, naphthyl, linear or branched C₁-C₇ alkyl, and mixtures thereof, and W units are spacer units which modulate the distance between the R⁴, R⁵ and —CHO units. Preferably, —RCHO has the formula:

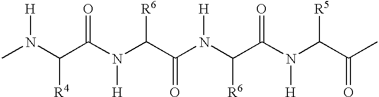

wherein each R⁶ is independently selected from the group consisting of hydrogen, methyl, hydroxymethyl, 1-hydroxylethyl, 1-methylethyl, 1-methylpropyl, 2-methylpropyl, benzyl, 4-hydroxyphenyl, 2-pyrrolidinyl, thiomethyl, (methylthio)methyl, carboxymethyl, carboxyethyl, 3-indolylmethyl, 4-aminobutyl, 3-guanidinopropyl, 1H-3-imidazolyl-, and mixtures thereof.

A preferred enzyme inhibitor conjugate has the formula:

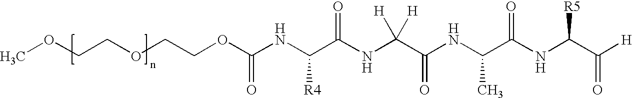

wherein R⁴ and R⁵ are each independently selected from the group consisting of substituted or unsubstituted: phenyl, benzyl, naphthyl, linear or branched C₁-C₇ alkyl, and mixtures thereof, and n is from 3 to 200.

A preferred enzyme inhibitor conjugate has the structure:

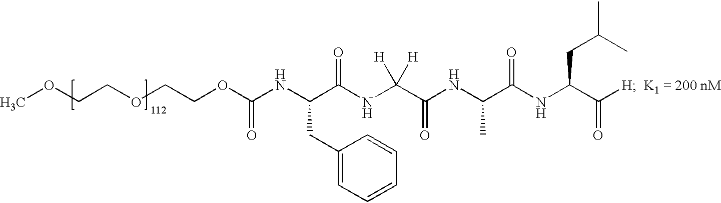

Suitable inhibitors for use herein also include variants of Streptomyces subtilisin inhibitor (SSI) and those inhibitors having at least 70% amino acid sequence homology to SSI (“SSI-like” inhibitors), wherein the variants comprise one or more of the following substitutions: A62K, L63I, M73P, D83C, S98D, S98E. Also suitable are polyethylene glycol-modified forms of SSI and SSI-like inhibitors and variants of the same.

Inhibitors that are not suitable for use herein because of their binding properties, include, but are not limited to Streptomyces subtilisin inhibitor (SSI) (Kᵢ<1 nM) and those inhibitors having at least 70% amino acid sequence homology to SSI (“SSI-like” inhibitors) lacking the substitutions identified above, and also inhibitors those conforming to the following structures:

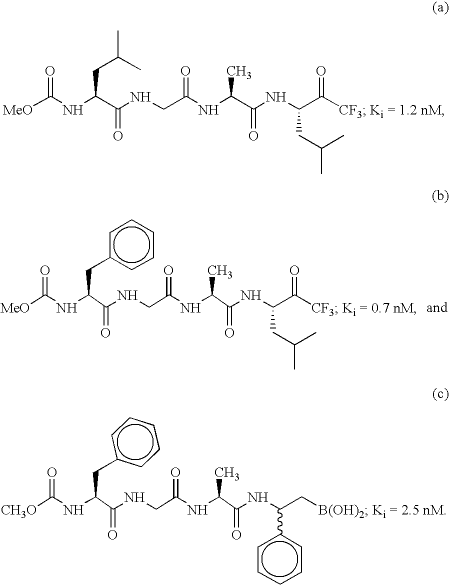

### II. Optional Components

The compositions of the present invention may, in some embodiments, further comprise additional optional components known or otherwise effective for use in topically applied personal care products, examples of which are described below. Any optional component(s) should be physically and chemically compatible with the essential components of the composition, and should not otherwise unduly impair product stability, aesthetics or performance.

A. Polyhydric Alcohol

The compositions of the present invention may additionally comprise a polyhydric alcohol in an amount that is sufficiently effective to enhance the stability of the enzyme and/or independently hydrate the skin. Typically a safe and effective amount will range from 0.5% to 40%, more preferably from 1% to 30%, more preferably from 2% to 20%, more preferably from 1% to 15%. The amounts of polyhydric alcohol and water may be selected such that the ratio (weight/weight %) of polyhydric alcohol to water typically will range. from 1:2 to 1:100, preferably from 1:3 to 1:50, more preferably from 1:5 to 1:25, still more preferably from 1:6 to 1:10. This may maximize the activity of the enzyme on skin. As used herein “polyhydric alcohol” means an organic compound comprising two, or more, alcohol functions or alkoxylated derivatives thereof. Typically, polyhydric alcohols suitable for use herein will have a number average molecular weight of less than 50,000, preferably less than 35,000, more preferably less than 2,000. When the inventive compositions are formulated as oil-in-water emulsion, preferably the polyhydric alcohol is present in the continuous phase.

Polyhydric alcohols suitable for use herein include, but are not limited to polyvinylalcohol (e.g. MOWIOL 4-88 (RTM) available from Clariant (Germany)), polyalkylene glycols, preferably alkylene polyols and their derivatives, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,2-butylene glycol and 1,3-butylene glycol), hexane triol (e.g., 1,2,6-hexanetriol), 2,4,4-trimethyl-pentanediol, neopentyl glycol, glycerine, ethoxylated glycerine, propane-1,3 diol, propoxylated glycerine, inositol, palatinit (isomalt), butane-1,4-diol, butoxytriol, and mixtures thereof. The alkoxylated derivatives of any of the above polyhydric alcohols are also suitable for use herein. Preferred polyhydric alcohols of the present invention are glycerine, polyethylene glycol (more preferably those having an average molecular weight of 200 to 400), hexane triol, butane-1,4-diol, butoxytriol, erythritol, xylitol, and mixtures thereof, more preferably glycerine, polyethylene glycol (more preferably those having an average molecular weight of 200 to 400), and mixtures thereof.

Also suitable are: (a) monosaccharides, e.g. ribose, ribulose, arabinose, xylose, xylulose, lyxose, allose, altrose, glucose, mannose, gulose, idose, galactose, talose, psicose, fructose, sorbose, tagatose, and mixtures thereof, (b) disaccharides, e.g. lactose, maltose, isomaltose, cellose, trehalose, sucrose, and mixtures thereof, (c) oligosaccharides, e.g. malto-oligosaccharide, cello-oligosaccharide, and mixtures thereof, and (d) mixtures thereof.

B. Osmo-Protectants

The compositions of the present invention may additionally comprise an osmo-protectant in an amount that is sufficiently effective to stabilize the enzyme, which may allow the amount of inhibitor used to be reduced. Typically a safe and effective amount will range from 1% to 50%, by weight of the composition, preferably from 3% to 25%, more preferably from 5% to 15%, still more preferably from 7% to 12%, yet more preferably from 7% to 10%. Both the protonated and de-protonated forms of the osmo-protectants described herein are suitable for use in the inventive compositions, the protonated forms being preferred. Typically there is an inverse relationship between an ingredient's solubility and hydrophobicity. Osmo-protectants suitable for use herein will typically have a solubility greater than 1% at pH 7 in water at 25° C. Also suitable for use herein are the osmo-protectants ectoin and hydroxyectoin.

Osmo-protectants that are suitable for use herein, include, but are not limited to:

(i) an osmo-protectant conforming to formula (1):

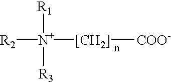

wherein R₁, R₂, and R₃ are independently selected from —H, —CH₃, —CH₂CH₃, and [—CH₂CH(OH)R₄, wherein R₄ is selected from —H, and C1 to C4 alkanes]; and

wherein n=an integer from 1 to 3, preferably 1;

preferably wherein (R₁, R₂, and R₃ are all —CH₃, and n is 1) or (R₁, R₂, and R₃ are all

—CH₃, and n is 3) or (R₁ and R₂ are —CH₃, R₃ is —H, and n is 1) or (R₁ is —CH₃, R₂ and R₃ are —H, and n is 1); or (R₁ and R₂ are —CH₂ (OH)R₄, R₄ is —H, R₃ is —H, and n is 1);

(ii) an osmo-protectant conforming to formula (I), wherein any two of R₁, R₂, and R₃ are independently selected from —H, and —CH₃, and the third moiety of R₁, R₂, and R₃ is selected from —H and —(CH₂)mCH₃ wherein m is 4 or 5; and wherein n=an integer from 1 to 3, preferably 1; preferably wherein (R₁ and R₂ are —CH₃, R₃ is —CH₂)mCH₃ wherein m is 5, and n is 1);

(iii) an osmo-protectant conforming to formula (II):

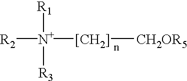

wherein R₁, R₂, R₃ R₄, and n are defined as in part (i) above; and wherein R₅ is selected from PO₃ and SO₃;

preferably wherein (R₁, R₂, and R₃ are —CH₃; n is 1; and R₅ is PO₃);

(iv) an osmo-protectant selected from the group consisting of alanine, glycine, serine, proline, carnitine, taurine, trimethylamineoxide, ectoin, and hydroxyectoin;

preferably selected from the group consisting of serine, proline, carnitine;

(v) an osmo-protectant selected from the group consisting of tricine, dimethyl proline, gamma-butyro betaine, beta-alanine betaine, valine betaine, lysine betaine, omithine betaine, alanine betaine, glutamic acid betaine, and phenyalanine betaine;

preferably selected from the group consisting of tricine and gamma-butyro betaine; and

(vi) mixtures thereof.

Non-limiting examples of preferred osmo-protectants for use herein are identified immediately above. Specific examples of preferred osmo-protectants include trimethylglycine hydrate, available as TEGOCARE AP (RTM), from T. H. Goldschmidt (Germany), proline, available as proline, from Huls-Degaussa (Germany), and bicine, available as bicine, from Sigma Chemical (USA).

C. Lyotropic Stabilizers

The inventive compositions may further comprise a lyotropic stabilizer in an amount that is sufficiently effective to enhance the stability of the enzyme. Typically a safe and effective amount will range from 0.1% to 5%, preferably from 0.5% to 3%, more preferably from 0.2% to 2%. The lyotropic stabilizers suitable for use herein may typically be incorporated in their salt form, wherein the cation is any monovalent ion, preferably one selected from the group consisting of alkaline metals, ammonium, tris[hydroxymethyl]aminomethane (TRIS), acetamide monoethanolamine (AMEA), and triethanolamine (TEA), more preferably selected from sodium, potassium, lithium, and acetamide monoethanolamine (AMEA).

Suitable lyotropic stabilizers for use herein include but are not limited to formate, acetate, propionate, glycolate, glycerate, malonate, succinate, adipate, malate, tartarate, sulfo-succinate, sulfate, phosphate, citrate, isethionate, glycerol phosphate, benzoate, glutarate, pimelate, suberate, phytate, and mixtures thereof; preferably formate, glycolate, adipate, malonate, sulfate, phosphate, succinate, and mixtures thereof.

Also suitable are: (a) oxidized monosaccharides, e.g. ribonic acid, ribulonic acid, arabinonic acid, xylonic acid, xylulonic acid, lyxonic acid, allonic acid, altronic acid, gluconic acid, mannonic acid, gulonic acid, idonic acid, galactonic acid, talonic acid, glucoheptonic acid, psiconic acid, fructonic acid, sorbonic acid, tagatonic acid, glucuronic acid, and mixtures thereof; (b) oxidized disaccharides, e.g. lactobionic acid, maltobionic acid, isomaltobionic acid, cellobionic acid, and mixtures thereof; (c) oxidized oligosaccharides, e.g. oxidized malto-oligosaccharide, oxidized cello-oligosaccharide, and mixtures thereof; (d) oxidized polysaccharides, e.g. oxidized cellulose; chitin; gum arabic; gum karaya; gum xanthan; oxidized gum guar; oxidized locust bean gum; oxidized agars; oxidized algins; oxidized gellan gum; and mixtures thereof; and (e) mixtures thereof.

D. Additional Enzyme Inhibitors

The inventive compositions may further comprise enzyme inhibitor in addition to those described above as essential to the invention, in an amount that is sufficiently effective to reduce the rate of auto-digestion of the enzyme, thereby enhancing the stability of the enzyme. Typically a safe and effective amount will range from 0.01% to 5%, preferably from 0.1% to 2%, more preferably from 0.5% to 1.5%.

Suitable additional inhibitors include but are not limited to boric acid Kᵢ>1,000,000 nM, butylboronic acid Kᵢ=7,200,000 nM, phenylboronic acid Kᵢ=160,000 nM, and aryl boronic acid derivatives according to the following formulae:

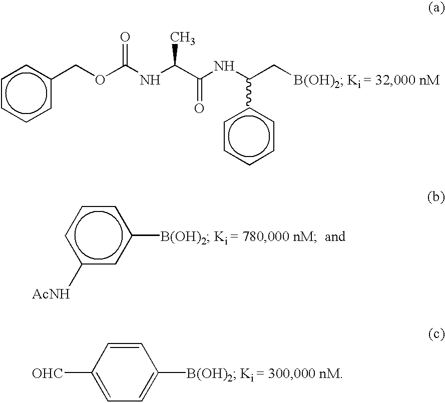

Suitable additional inhibitors for use herein also include inhibitors conforming to the following formulae:

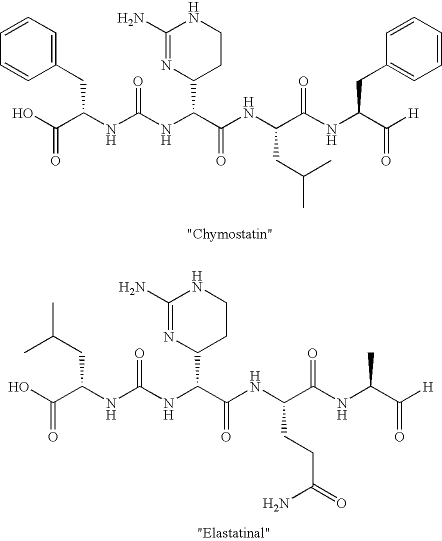

Also suitable for use herein are other naturally derived inhibitors, e.g. leupeptide Kᵢ=230,000 nM.

E. pH Modifiers and Buffering Agents

The inventive compositions may further comprise a pH modifiers in an amount that is sufficiently effective to adjust the pH of the composition to fall within a range from 5.5 to 9, preferably from 6 to 8.5, more preferably from 6.5 to 8. One skilled in the art will understand that the amount of pH modifier to be added will be dependent on the quantity and type of other ingredients selected to make the compositions described herein.

pH modifiers that are suitable for use herein include, but are not limited to: hydroxides; preferred are the sodium salts of these. In addition, the inventive compositions may further comprise buffering agents in an amount that is sufficiently effective to minimize pH drift. Buffering agents that are suitable for use herein include, but are not limited to histidine, 1,4-piperizinediethanesulfonic acid (PIPES), tris[hydroxymethyl]aminomethane (TRIS), [bis-(2-hydroxyethyl)imino]-tris-[(hydroxymethyl)methane] (BIS-TRIS), 1-[4-(2-hydroxyethyl)-1-piperazinyl]ethane-2-sulfonic acid (HEPES), N-(2-acetamido)-2-iminodiacetic acid (ADA), 2-[(2-amino-2-oxoethyl)amino]ethanesulfonic acid (ACES), N,N-bis(2-hydroxethyl)-taurine (BES), morpholinopropanesulfonic acid (MOPS), N-[2-hydroxy-1,1-bis(hydromethyl)ethyl]-taurine (TES), 3-[bis(2-hydroxyethyl)amino]-2-hydroxypropane sulphonic acid (DIPSO), and (salts of phosphate, pyrophosphate, citrate, and mellitate), and mixtures thereof, preferably salts of phosphate.

F. Polymeric thickeners

The inventive compositions may further comprise a polymeric thickener in an amount that is sufficiently effective to provide delightful in-use experience for a consumer. Typically a safe and effective amount will range from 0.1% to 10%, preferably from 0.5% to 8%, more preferably from 1% to 5%. Preferred thickeners for use herein will have an increased electrolyte sufficient to provide the compositions containing electrolytes with a minimal viscosity loss. Typically, the compositions described herein will have a viscosity greater than 4,000 mPa.s (measured, e.g. by Brookfield viscometer DVII+Spindle C heliopath 5 rpm at 25° C.). Polymeric thickeners suitable for use herein will have a number average molecular weight of greater than 50,000, preferably greater than 100,000.

Preferred polymeric thickeners for use herein include but are not limited to non-ionic thickening agents and anionic thickening agents, and mixtures thereof. Suitable non-ionic thickening agents include polyacrylamide polymers, crosslinked poly(N-vinylpyrrolidones), polysaccharides, natural or synthetic gums, polyvinylpyrrolidone, and polyvinylalcohol (e.g. MOWIOL 40-88 (RTM) available from Clariant (Germany)). Suitable anionic thickening agents include acrylic acid/ethyl acrylate copolymers, carboxyvinyl polymers and crosslinked copolymers of alkyl vinyl ethers and maleic anhydride. Preferred thickening agents for use herein are the polyacrylamide/AMPS polymers such as polyacrylamide and isoparaffin and laureth-7, available as SEPIGEL 305 (RTM) from Seppic Corporation, and acrylic acid/ethyl acrylate copolymers and the carboxyvinyl polymers, including hydrophobically modified derivatives thereof, available as CARBOPOL (RTM) resins from Noveon (Cleveland, Ohio). Still other suitable resins are described in WO 98/22085. Also, preferred are acrylic acid/acrylamide polymers, preferably those having an acrylic acid to acrylamide ratio from 4:1 to 1000:1, and produced via an emulsion polymerisation process, one such polymer being ammonium acrylates/acrylamide crosspolymer, available as EX-617 (RTM) from Noveon (Cleveland, Ohio), and hydroxyethyl acrylate/AMPS polymers, available as SEPIGEL NS (RTM), from Seppic Corporation. Also suitable for use herein are gums, e.g. xanthan, karaya, gellan, wellan, arabic, carrageanan, biosaccharide gum-1 (e.g. FUCOGEL 1000 (RTM), available from Solabia (France)), and locust bean. Also suitable are alginate and agarose.

G. Skin Active Agents

The compositions of the present invention may comprise a safe and effective amount of a skin active agent. When present, such agents will typically be included in an amount ranging from 0.1% to 20%, preferably from 1% to 10%, more preferably from 2% to 8%.

Some preferred skin active agents for use herein include but are not limited to a vitamin B₃ compound, panthenol, vitamin E, tocopherol acetate, retinol, retinyl propionate, retinyl palmitate, retinoic acid, vitamin C, vitamin D, caffeine, theobromine, allantoin, alpha-hydroxycarboxylic acids (e.g. glycolic acid, lactic acid, 2-hydroxyoctanoic acid), beta-hydroxycarboxylic acids (e.g. salicylic acid), (alpha- and beta- hydroxycarboxylic acids in combination with salts of glycyrrhic acid, alpha-bisabol, and triclosan), farnesol, phytantriol, glucosamine, magnesium ascorbyl phosphate, ascorbyl glucoside, pyridoxine, palmityl penta-peptide-3 (available as MATRIXYL (RTM) from Sederma Company), pitera (yeast extract), phytosterols (e.g. stigmasterol, sitosterol, brassicasterol, campesterol), flavanoids (e.g. chalcones, chromones, flavones, isoflavones), inhibitors of HMG-coA-reductase (e.g. mevastatin, lovastatin), and mixtures thereof, including any of those described when delivered inside of nanocapsules. A preferred combination of skin active agents is a complex comprising niacinamide, panthenol and a retinol compound. Other suitable skin active agents are described herein.

The compositions of the present invention may comprise a safe and effective amount of a vitamin B3 compound. Vitamin B₃ compounds are particularly useful for regulating skin condition as described in WO 97/39733A1, published Oct. 30, 1997. When vitamin B₃ compounds are added, typical amounts will range from 0.1% to 10%, more preferably from 0.5% to 8%, more preferably from 1% to 5%, and still more preferably from 2% to 5%. Suitable vitamin B₃ compounds for use herein include but are not limited to compounds having the formula:

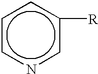

wherein R is —CONH₂ (i.e., niacinamide), —COOH (i.e., nicotinic acid) or —CH₂OH (i.e., nicotinyl alcohol); derivatives thereof, and salts of any of the foregoing. Exemplary non-limiting derivatives of the above vitamin B3 compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

The compositions of the present invention may also comprise a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), more preferably retinoids other than retinoic acid. Such compounds are known and are commercially available from, e.g., Sigma Chemical Company (St. Louis, Mo.), and Boerhinger Mannheim (Indianapolis, Ind.). Other retinoids which are useful herein are described in U.S. Pat. Nos. 4,677,120, issued Jun. 30, 1987 to Parish et al.; 4,885,311, issued Dec. 5, 1989 to Parish et al.; 5,049,584, issued Sep. 17, 1991 to Purcell et al.; 5,124,356, issued Jun. 23, 1992 to Purcell et al.; and Reissue 34,075, issued Sep. 22, 1992 to Purcell et al. Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]nicotinate). Preferred retinoids are retinol, retinyl palmitate, retinyl acetate, retinyl propionate, retinal and combinations thereof. When present, retinoids will typically be in amounts ranging from 0.005% to 2%, preferably from 0.01% to 2%. Retinol is preferably used in an amount from 0.01% to 0.15%; retinol esters are preferably used in an amount from 0.01% to 2%; retinoic acids are preferably used in an amount from 0.01% to 0.25%; tocopheryl-retinoate, adapalene, and tazarotene are preferably used in an amount from 0.01 % to 2%.

H. Oil Phase

The compositions of the present invention may be formulated as an emulsion, comprising one or more oil phases in one or more aqueous phases, each oil phase comprising a single oily component or a mixture of oily components in miscible or homogenous form. The total level of oil phase components will typically be from 0.1% to 60%, preferably from 1% to 30%, more preferably from 1% to 10%, still more preferably from 2% to 10%. When present, the oil phase(s) may comprise preferably comprise an emollient, a silicone oil, or mixtures thereof; they may also comprise additional oily components such as a natural or synthetic oils selected from mineral, vegetable, and animal oils (e.g. lanolin), fats and waxes, fatty acid esters, fatty alcohols, fatty acids and mixtures thereof. Preferred for use herein are for example, saturated and unsaturated fatty alcohols such as behenyl alcohol, cetyl alcohol and stearyl alcohol and hydrocarbons such as mineral oils or petrolatum. Further examples suitable for use herein are disclosed in WO98/22085. Preferred embodiments may comprise from 0.1% to 5% of an unsaturated fatty acid or ester as described in WO98/22085.

I. Emollient

The compositions of the present invention may comprise emollients, the amount when present typically ranging from 0.1% to 10%, preferably from 0.1% to 8%, more preferably from 0.5% to 5%.

Suitable emollient materials include branched chain hydrocarbons having an weight average molecular weight of from about 100 to about 15,000, preferably from about 100 to 1000; compounds conforming to the formula:

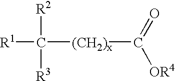

wherein R¹ is selected from —H or —CH₃, R², R³ and R⁴ are independently selected from C1 to C20 straight chain or branched chain alkyl, and x is an integer of from 1-20. Suitable ester emollient materials of this formula include but are not limited to: methyl isostearate, isopropyl isostearate, isostearyl neopentanoate. isononyl isononanoate, isodecyl octanoate, isodecyl isononanoate, tridecyl isononanoate, myristyl octanoate, octyl pelargonate, octyl isononanoate, myristyl myristate, myristyl neopentanoate, myristyl octanoate, myristyl propionate, isopropyl myristate and mixtures thereof.

Suitable emollients for use herein also include compounds conforming to the formula:

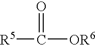

wherein R⁵ is selected from optionally hydroxy or C1 to C4 alkyl substituted benzyl and R⁶ is selected from C1 to C20 branched or straight chain alkyl; and mixtures thereof. Suitable ester emollient materials of this formula include but are not limited to C12-15 alkyl benzoates.

Suitable emollients for use herein also include vegetable oils and hydrogenated vegetable oils, e.g. safflower oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, meadowfoam seed oil, shea butter, partially and fully hydrogenated oils from the foregoing sources, and mixtures thereof.

Suitable branched chain hydrocarbon emollients for use herein include but are not limited to isododecane, isohexadecane, isoeicosane, isooctahexacontane, isohexapentacontahectane, isopentacontaoctactane, and mixtures thereof, e.g. those branched chain aliphatic hydrocarbons sold under the trade name PERMETHYL (RTM), available from Presperse (New Jersey).

Other suitable emollients for use herein are perhydroxysqualene, perflouropolyethers, methylglucosesesquistearate, tributylcitrate, glycerol stearylcitrate, butylene glycol dicaprylatedicaprate, capric caprylic triglyceride, and mixtures thereof.

Preferred emollients for use herein are isohexadecane, isooctacontane, isononyl isononanoate, isodecyl octanoate, isodecyl isononanoate, tridecyl isononanoate, myristyl octanoate, octyl isononanoate, myristyl myristate, methyl isostearate, isopropyl isostearate, C12-15 alkyl benzoates and mixtures thereof, more preferably isohexadecane, isononyl isononanoate, methyl isostearate, isopropyl isostearate, and mixtures thereof.

The compositions of the present invention may further comprise as an additional emollient, a polyol carboxylic acid ester, typically in amounts ranging from 0.01% to 20%, preferably from 0.1% to 15%, more preferably from 0.1% to 10%. The level of polyol ester by weight of the oil in the composition is typically from 1% to 30%, preferably from 5% to 20%. The weight ratio of the carboxylic acid polyol ester to the aforementioned emollient materials will typically range from 5:1 to 1:5, preferably from 2:1 to 1:2. Preferred polyol polyesters for use herein are C1 to C30 mono- and poly- esters of sugars and related materials, e.g. cottonseed oil or soybean oil fatty acid esters of sucrose, and those materials described in WO 96/16636; more preferred is a material known by the INCI name sucrose polycottonseedate.

J. Silicone Oil

The compositions of the present invention may comprise at least one silicone oil phase, typically the oil comprising from 0.1% to 20%, preferably from 0.5% to 10%, more preferably from 0.5% to 5%. Silicone components may be fluids, including straight chain, branched and cyclic silicones. Suitable silicone fluids useful herein include silicones inclusive of polyalkyl siloxane fluids, polyaryl siloxane fluids, cyclic and linear polyalkylsiloxanes, polyalkoxylated silicones, amino and quaternary ammonium modified silicones, polyalkylaryl siloxanes or a polyether siloxane copolymer and mixtures thereof. The silicone fluids may be volatile or non-volatile. Silicone fluids generally have a weight average molecular weight of less than 200,000. Suitable silicone fluids have a molecular weight of 100,000 or less, preferably 50,000 or less, more preferably 10,000 or less. Preferably the silicone fluid is selected from silicone fluids having a weight average molecular weight in the range from 100 to 50,000 and preferably from 200 to 40,000.

Typically, silicone fluids have a viscosity ranging from about 0.65 to about 600,000 mm².s⁻¹, preferably from about 0.65 to about 10,000 mm².s^{31 1} at 25° C. The viscosity can be measured by means of a glass capillary viscometer as set forth in Dow Corning Corporate Test Method CTM0004, Jul. 29, 1970. Suitable polydimethyl siloxanes that can be used herein include those available, for example, from the General Electric Company as the SF and VISCASIL (RTM) series and from Dow Corning as the DOW CORNING 200 (RTM) series. Also useful are essentially non-volatile polyalkylarylsiloxanes, for example, polymethyl-phenylsiloxanes, having viscosities of about 0.65 to 30,000 mm².s⁻¹ at 25° C. These siloxanes are available, for example, from the General Electric Company as SF 1075 (RTM) methyl phenyl fluid or from Dow Corning as 556 COSMETIC GRADE FLUID (RTM). Cyclic polydimethylsiloxanes suitable for use herein are those having a ring structure incorporating from about 3 to about 7 (CH₃)₂SiO moieties. Preferably, the silicone fluid is selected from dimethicone, decamethylcyclopentasiloxane, octamethylcyclotetrasiloxane, phenyl methicone, and mixtures thereof.

Silicone gums may also be used herein. The term “silicone gum” herein means high molecular weight silicones having a weight average molecular weight in excess of 200,000 and preferably from 200,000 to 4,000,000. Included are non-volatile polyalkyl and polyaryl siloxane gums. In preferred embodiments, a silicone oil phase comprises a silicone gum or a mixture of silicones including the silicone gum. Typically, silicone gums have a viscosity at 25° C. in excess of 1,000,000 mm²s⁻¹. The silicone gums include dimethicones as described in Noll, Walter, Chemistry and Technology of Silicones, New York: Academic Press 1968. Also describing silicone gums are General Electric Silicone Rubber Product Data Sheets SE 30, SE 33, SE 54 and SE 76. Specific examples of silicone gums include polydimethylsiloxane, (polydimethylsiloxane)(methylvinylsiloxane) copolymer, poly(dimethylsiloxane)(diphenyl)-(methylvinylsiloxane) copolymer and mixtures thereof. Preferred silicone gums for use herein are silicone gums having a molecular weight of from 200,000 to 4,000,000 selected from dimethiconol, and dimethicone and mixtures thereof.

A silicone phase herein preferably comprises a silicone gum incorporated into the composition as part of a silicone gum-fluid blend. When the silicone gum is incorporated as part of a silicone gum-fluid blend, the silicone gum typically constitutes from 5% to 40%, preferably from 10% to 20% by weight of the silicone gum-fluid blend. Suitable silicone gum-fluid blends herein are mixtures comprising:

(i) a silicone having a molecular weight of from 200,000 to 4,000,000 selected from dimethiconol, fluorosilicone and dimethicone and mixtures thereof; and

(ii) a carrier which is a silicone fluid, the carrier having a viscosity from 0.65 mm².s⁻¹ to 100 mm².s⁻¹,

wherein the ratio of i) to ii) is from 10:90 to 20:80 and wherein said silicone gum-based component has a final viscosity of from 100 mm².s⁻¹ to 100,000 mm².s-¹, preferably from 500 mm².s⁻¹ to 10,000 mm².s⁻¹.

A preferred silicone-gum fluid blend based component for use in the compositions herein is a dimethiconol gum having a molecular weight of from 200,000 to 4,000,000 along with a silicone fluid carrier with a viscosity of about 0.65 to 100 mm².s⁻¹. Examples of these silicone components are DOW CORNING Q2-1403 (RTM) (85% 5 mm².s⁻¹ Dimethicone Fluid/15% Dimethiconol) and DOW CORNING Q2-1402 (RTM) available from Dow Corning.

Further silicone components suitable for use in a silicone oil phase herein are crosslinked polyorganosiloxane polymers, optionally dispersed in a fluid carrier. In general, when present the crosslinked polyorganosiloxane polymers, together with its carrier (if present) comprises 0.1% to 20%, preferably from 0.5% to 10%, more preferably from 0.5% to 5%. Such polymers comprise polyorganosiloxane polymers crosslinked by a crosslinking agent. Suitable crosslinking agents are disclosed in W)98/22085. Examples of suitable polyorganosiloxane polymers-for use herein include methyl vinyl dimethicone, methyl vinyl diphenyl dimethicone and methyl vinyl phenyl methyl diphenyl dimethicone.

Specific commercially available crosslinked polyorganosiloxane polymers for use herein are silicone vinyl crosspolymer mixtures available under the tradename KSG (RTM) supplied by Shinetsu Chemical Co., Ltd, for example KSG-15, KSG-16, KSG-17, KSG-18 and DC9040 (RTM) from Dow Corning. These materials contain a combination of crosslinked polyorganosiloxane polymer and silicone fluid. Preferred for use herein in combination with the organic amphiphilic emulsifier material is KSG-18 (RTM). The assigned INCI names for KSG-15, KSG-16, KSG-17 and KSG-18 are cyclomethicone dimethicone/vinyl dimethicone crosspolymer, dimethicone dimethicone/vinyl dimethicone crosspolymer, cyclomethicone dimethicone/vinyl dimethicone crosspolymer and phenyl trimethicone dimethicone/phenyl vinyl dimethicone crosspolymer, respectively.

Another class of silicone components suitable for use in a silicone oil phase herein includes polydiorganosiloxane-polyoxyalkylene copolymers containing at least one polydiorganosiloxane segment and at least one polyoxyalkylene segment. Suitable polydiorganosiloxane segments and copolymers thereof are disclosed in WO98/22085. Suitable polydiorganosiloxane-polyalkylene copolymers are available commercially under the tradenames BELSIL (RTM) from Wacker-Chemie (Germany), and ABIL (RTM) from Goldschmidt (England), e.g. BELSIL 6031 (RTM) and ABIL B88183 (RTM). A preferred copolymer fluid blend for use herein includes Dow Corning's DC5225C (RTM) which has the CTFA designation Dimethicone/Dimethicone copolyol. Also useful are decyl methicone, octylmethicone, C16 to C18 methicone, available as SF1632 (RTM) from General Electric Company (USA).

K. Emulsifier/Surfactant

The inventive compositions may further comprise an emulsifier and/or surfactant, generally to help disperse and suspend the disperse phase within the continuous aqueous phase (when formulated as an emulsion). As used herein emulsifiers will be referred to collectively with surfactants.

Many suitable surfactants for use herein are described in WO 00/24372. A preferred surfactant of this type is decyl glucoside (the product obtained from the condensation of decyl alcohol with a glucose polymer). Decyl glucoside may optionally be incorporated into the inventive compositions described herein. While not being bound by theory, it is believed that decyl glucoside may improve contact between the enzyme and the substrate (skin) by allowing more efficient wetting to take place. In leave-on formulations, decyl glucoside may be advantageously used to perform this function where other surfactants, typically anionic ones, may cause unacceptable irritation to the skin. Decyl glucoside has been found to not increase irritation of the skin when used in a leave-on formulation.

Suitable nonionic surfactants include (polyethylene oxide/polypropylene oxide) copolymer and (polyethylene oxide/polybutylene oxide) copoloymer, such as that available from BASF (Germany) under the tradename PLURONIC, particularly PLURONIC L92.

Suitable surfactants are non-ionic, e.g. condensation products of long chain alcohols, e.g. C₈₋₃₀ alcohols, with sugar or starch polymers, e.g. glycosides. Other useful nonionic surfactants include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from 6 to 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂-(i.e. derived from ethylene glycol or oxide) or —OCH₂CHCH₃-(i.e. derived from propylene glycol or oxide), and n is an integer from 6 to 100. A preferred emulsifier for use herein is a fatty acid ester blend based on a mixture of sorbitan fatty acid ester and sucrose fatty acid ester, e.g. a blend of sorbiton stearate and sucrose cocoate. This is commercially available from ICI as ARLATONE 2121 (RTM). Even further suitable examples include a mixture of cetearyl alcohols, cetearyl glucosides such as those available as MONTANOV 68 (RTM) from Seppic and EMULGADE PL68/50 (RTM) available from Henkel.

Hydrophilic surfactants useful herein may alternatively or additionally include any of a variety of cationic, anionic, zwitterionic, and amphoteric surfactants known in the art (see McCutcheon's , Detergents and Emulsifiers, North American Edition (1986), published by Allured Publishing Corporation). Useful herein also are alkoyl isethionates (e.g., C₁₂-C₃₀), alkyl and alkyl ether sulfates and salts thereof, alkyl and alkyl ether phosphates and salts thereof, alkyl methyl taurates (e.g., C₁₂- C₃₀), and soaps (e.g., alkali metal salts, e.g., sodium or potassium salts) of fatty acids, and sugar and aminosugar (e.g. N-butanoyl-D-glucosamine and 6-O-octanoyl-D-maltose).

When formulated as an emulsion, the compositions of the present invention may include a silicone containing surfactant, e.g. organically modified organopolysiloxanes, also known as silicone surfactants. Useful silicone emulsifiers include dimethicone copolyols. These materials are polydimethyl siloxanes that have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, e.g. compounds that contain C2 to C30 pendant side chains. Still other useful dimethicone copolyols include materials having various cationic, anionic, amphoteric, and zwitterionic pendant moieties.

L. Suspending Agent

The compositions herein preferably also include one or more suspending agents. Suitable suspending agents for use herein include any of several long chain acyl derivative materials or mixtures of such materials. Included are ethylene glycol esters of fatty acids having from 16 to 22 carbon atoms. Preferred are the ethylene glycol stearates, both mono and distearate, but particularly the distearate containing less than about 7% of the mono stearate. Other suspending agents found useful are alkanol amides of fatty acids, having from 16 to 22 carbon atoms, preferably from 16 to 18 carbon atoms. Preferred alkanol amides are stearic monoethanolamide, stearic diethanolamide, stearic monoisopropanolamide and stearic monoethanolamide stearate.

The suspending agent is preferably present at a level from 0.1% to 5%, preferably from 0.1% to 3%. The suspending agent serves to assist in suspending the water-insoluble oil and may give pearlescence to the product. Mixtures of suspending agents are also suitable for use herein.

M. Anti-Microbial and Anti-Fungal Actives

Optional anti-microbials and anti-fungal actives suitable for use herein include but are not limited to: beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetra-cycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine, hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, clotrimazole, cetylpyridinium chloride (CPC), piroctone olamine, selenium sulfide, ketoconazole, triclocarbon, triclosan, triclocarban (also known as trichlorocarbanilide), hexachlorophene, (3,4,5-tribromosalicylanilide), zinc pyrithione, itraconazole, asiatic acid, hinokitiol, mipirocin and those described in EP 680,745, clinacycin hydrochloride, benzoyl peroxide, benzyl peroxide, minocyclin, phenoxy isopropanol, and mixtures thereof

N. Sunscreens

Compositions of the present invention may comprise an organic sunscreen having UVA absorbing properties, UVB absorbing properties or a mixture thereof. Preferred sunscreens include but are not limited to dibenzoylmethane and derivatives thereof, e.g. 4-(1,1-dimethylethyl)-4′-methoxydibenzoylmethane; 4-isoproplydibenzoylmethane; p-aminobenzoic acid, oxybenzone, homomenthyl salicylate, octyl salicylate, cinnamates and their derivatives, e.g. 2-ethylhexyl-p-methoxycinnamate and octyl-p-methoxycinnamate, TEA salicylate, octyldimethyl PABA, camphor derivatives and their derivatives, and mixtures thereof. In addition to the organic sunscreens compositions of the present invention may comprise inorganic physical sunblocks, preferably zinc oxide and titanium dioxide, and mixtures thereof, which may be uncoated or coated with a variety of materials including but not limited to amino acids, aluminium compounds such as alumina, aluminium stearate, and aluminium laurate; carboxylic acids and their salts e.g. stearic acid; phospholipids such as lecithin; organic silicone compounds; inorganic silicone compounds such as silica and silicates; and mixtures thereof. A preferred titanium dioxide is commercially available from Tayca (Japan) and is distributed by Tri-K Industries (New Jersey) under the MT micro-ionised series (e.g. MT 100SAS (RTM)).

O. Particulate Matter

Compositions of the present invention may comprise pigments that, where water-insoluble, contribute to and are included in the total level of oil phase ingredients. Pigments suitable for use herein may organic and/or inorganic. Also included within the term pigment are materials having a low color or luster such as matte finishing agents, and also light scattering agents. Preferably the compositions of the present invention comprise particulate materials having a refractive index of from 1.3 to 1.7, the particulate materials being dispersed in the composition and having a median particle size of from 2 microns to 30 microns. Preferably the particulates useful herein have relatively narrow distributions, by which is meant that more than 50% of the particles fall within 3 microns either side of the respective median value. Also preferred is that more than 50%, preferably more than 60%, more preferably more than 70% of particles fall within the size ranges prescribed for the respective median values. Suitable particulate materials are organic or organosilicone and preferably organosilicone polymers.

Preferred particles are free-flowing, solid, materials. By “solid” is meant that the particles are not hollow. The void at the centre of hollow particles can have an adverse effect on refractive index and therefore the visual effects of the particles on either skin or the composition. Suitable organic particulate materials include those made of polymethylsilsesquioxane, polyamide, polythene, polyacrylonitrile, polyacrylic acid, polymethacrylic acid, polystyrene, polytetrafluoroethylene (PTFE), Nylon 12 (e.g. ORGASOL 2002 NAT COS D (RTM) from Elf Atochem, and NYLON POLYWL10, available from Optima (England)) and poly(vinylidene chloride). Copolymers derived from monomers of the aforementioned materials may also be used. Inorganic materials include silica and boron nitride. Representative commercially available examples of useful particulate materials herein are TOSPEARL 145 and TOSPEARL 2000 (RTMs) available from GE Silicones (New York) which have a median particle size of 4.5 microns and EA-209 (RTM), available from Kobo Products (USA) which is an ethylene/acrylic acid copolymer having a median particle size of 10 microns. Further examples of suitable pigments are titanium dioxide, pre-dispersed titanium dioxide, e.g. GWL75CAP (RTM) available from Kobo Products (USA), iron oxides, acyglutamate iron oxides, ultramarine blue, D&C dyes, carmine, and mixtures thereof, and aluminium starch octenylsuccinate (available as DRY FLO (RTM) from National Starch & Chemical Ltd). The pigments may also be treated with compounds such as amino acids, silicones, lecithin and ester oils.

P. Other Optional Components

Other suitable optional ingredients include but are not limited to: anti-static agents; foam boosters (e.g. fatty ester (e.g. C₈-C₂₂) mono- and di (C₁-C₅, especially C₁-C₃) alkanol amides, preferably coconut monoethanolamide, coconut diethanolamide, and mixtures thereof), viscosity modifiers and thickeners (e.g. sodium chloride, sodium sulfate, and magnesium sulfate); pearlescent aids; perfumes; preservatives, e.g. DMDM hydantoin, benzalkonium chloride, methyl paraben, propyl paraben, and benzyl alcohol; anti-dandruff actives, e.g. pyridinethione salts, selenium sulfide, particulate sulfur, and mixtures thereof, biological additives, bulking agents, chelating agents (e.g. ethylenediamine tetraacetic acid (EDTA), and furildioxime); suspending agents, e.g. alkyl (C₁₆-C₂₂) dimethyl amine oxides such as stearyl dimethyl amino oxide and trihydroxystearin commercially available as THIXIN (RTM) from Rheox.(USA); other additives, colorants, cosmetic astringents, cosmetic biocides, denaturants, drug astringents, skin lightening agents, external analgesics, film formers, opacifying agents, reducing agents, skin bleaching agents, anti-inflammatory agents; anti-oxidants/radical scavengers; and other ingredients known for use in personal care compositions such as those found in the CTFA International Cosmetic Ingredient Dictionary and Handbook, eighth edition, volume 2, edited by Wenninger and McEwen (The Cosmetic, Toiletry, and Fragrance Association, Inc., Washington, D.C., 1999).

### Methods of Manufacture

The compositions of the present invention may be synthesized using any conventional method. Generally, the aqueous phase, and when present, the oil phase would be prepared separately, with materials of similar phase partitioning being added in any order. If the final product is an emulsion, the two phases will then be combined with vigorous stirring. Any ingredients in the formulation with high volatility, or which are susceptible to hydrolysis at high temperatures, can be added with gentle stirring towards the end of the process, post emulsification if applicable. Typical methods suitable for use herein are described in WO 00/71093 and WO 00/48569, (the preceding particularly relevant to formulation as a body lotion) and WO 01/02477 (relevant to formulation as a gel patch) (all Procter & Gamble).

Such compositions can be in any form that delivers a safe and effective amount of the enzyme and/or any other skin active agent; suitable forms including but not limited to cream, gel, hydrogel, gel or hydrogel patch, mask (including face-mask), lotion, leave-on rinse, tonic, spray, ointment, poultice, foam, mousse, pomade, and paste, notably, the forms should be formulated as a “leave-on” product, however, a “rinse-off” product that is not to be rinsed off within a few minutes of application, may also be suitable. The inventive compositions may also be delivered and/or packaged with or via a substrate, e.g. an applicator brush, pad, wipe, cloth, towelette, sponge, puff, scrubber, and the like.

While typically, and even preferably, the compositions described herein will be formulated in a single container, they may alternatively be packaged in a kit comprising multiple containers, e.g. separate containers to be used separately upon or shortly prior to application, or in separate containers that are integrated, e.g. dual packaging, to deliver their respective compositions simultaneously. For example, one compartment may contain the enzyme, water, and certain Kᵢ inhibitor, and another compartment may contain certain skin active agents that are incompatible, e.g. for stability or pH reasons, with the contents of the first container. Another example would find one article of a kit comprising the enzyme, water, and certain Kᵢ inhibitor, and one or more individually packaged components containing other personal care compositions, e.g. sunscreens, or additional moisturizers, make-up removers, etc., useful in pre-treatment or post-treatment steps of a regimen that involves the first mentioned container, the regimen being useful for addressing specific needs of a consumer.

### Methods of Use

The method of the present invention involves the administration of the compositions described herein for improving skin feel and appearance. Such compositions are to be administered topically to the skin and/or hair, e.g. in order to reach the scalp skin from which the hair protrudes. The amount of the composition and the frequency of application to skin can vary depending on the desired effect and/or personal needs. The compositions of the present invention may be used as a pre- or post-treatment step to additional skin care processes taking place in order to further enhance skin feel and appearance. Where suitable, a consumer may be instructed to “wipe-off” as opposed to wash or rinse-off the inventive compositions after an appropriate amount of time has lapsed, following topical application.

### EXAMPLES

The following are non-limiting examples of embodiments of the present invention. The examples are given solely for the purpose of illustration and are not to be construed as limitations of the present invention, as many obvious and/or equivalent variations thereof are possible without departing from the spirit and scope of the invention, which would be recognized by one of ordinary skill in the art. In the examples, all concentrations are listed as weight percent, unless otherwise specified. As is apparent to one of ordinary skill in the art, the selection of ingredients will vary depending on the physical and chemical characteristics of the particular ingredients selected to make the present invention as described herein. The compositions of the examples may be made using conventional methods, as indicated above.

In all of the examples, Protease 1 is a variant of subtilisin BPN′ having a combination of amino acid substitutions at N76D-I122A-Y217L; Protease 2 is subtilisin BPN′, and Protease 3 is subtilisin Carlsberg. Inhibitor 1 is an enzyme inhibitor conjugate having the structure:

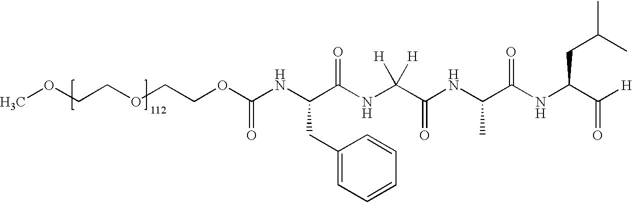

Inhibitor 2 is a variant of SSI comprising amino acid substitution A62K-L63I-M73P-D83C-S98E. Inhibitor 3 is a boronic acid derivative having the structure:

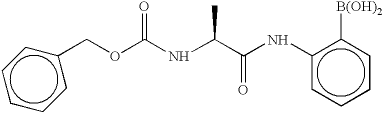

Examples I to XI may be formulated as body lotions, or face/body/foot cream moisturizers:

| | I | II | III | IV | V | VI | VII | VIII | VIIII | X | XI |
|---|---|---|---|---|---|---|---|---|---|---|---|
| Inhibitor 1 | 0.2 | 0.5 | | | | | 0.2 | | | 0.75 | 0.1 |
| Inhibitor 2 | | | 0.5 | 0.15 | | | | 0.7 | | | |
| Inhibitor 3 | | | | | 0.5 | 0.2 | | | 0.5 | | |
| Trimethyl glycine | | | 10 | 5 | | 8 | | | | | 5 |
| Sodium formate | | 1.0 | | 1.5 | | 0.5 | | | | 1.0 | |
| Formyl phenyl | | | 1.0 | | | | | | | | 0.5 |
| boronic acid | | | | | | | | | | | |
| Calcium chloride | 0.01 | | 0.01 | 0.01 | 0.01 | | 0.01 | 0.01 | 0.01 | | 0.01 |
| dihydrate | | | | | | | | | | | |
| Glycerin | 10 | | 5 | 1.5 | 5 | 10 | | 4 | 10 | 3 | |
| Polyethylene | | | | | 5 | | | 4 | | 7 | 2 |
| glycol 200 | | | | | | | | | | | |
| Erythritol | | | | | | 2 | | 4 | | | 2 |
| Polyacryamide/ | 2.5 | | 2.0 | 5.0 | | 3.0 | 2.5 | 2.0 | | 3.0 | 3.5 |
| isoparrafin/ | | | | | | | | | | | |
| laureth-7 | | | | | | | | | | | |
| Ammonium | | 1.5 | | | 2.0 | | | | 1.5 | | |
| polyacrylate/ | | | | | | | | | | | |
| Acrylamide | | | | | | | | | | | |
| polyethylene | | | | | | | | 0.8 | | | |
| oxide/polypropylene | | | | | | | | | | | |
| oxide | | | | | | | | | | | |
| copolymer | | | | | | | | | | | |
| Niacinamide | 3.5 | | 5.0 | | | 2.0 | | | | 1.0 | |
| Panthenol | 0.5 | 1.0 | | | | 1.0 | | | | 1.0 | |
| Tocopherol | | 2.0 | | | | | 2.0 | 5.0 | | | |
| nicotinate | | | | | | | | | | | |
| Retinyl propionate | | 0.1 | | 0.1 | | | 0.1 | | | 0.1 | |
| Tocopherol | 0.25 | | | | | 0.25 | | | | 1.0 | |
| acetate | | | | | | | | | | | |
| Octylmethoxycinna- | | | | | 2.0 | | | | | | |
| mate | | | | | | | | | | | |
| Polydimethyl- | | 2.0 | | | 1.0 | | 1.0 | 0.25 | | 2.0 | |
| silsequioxane | | | | | | | | | | | |
| Nylon 12 | | | 2.0 | | 1.0 | | 1.0 | 2.0 | | | |
| Titanium dioxide | | | | 0.1 | 0.4 | | | | 0.55 | | |
| Cetearyl glucoside | 0.1 | 0.1 | 0.2 | 0.5 | | 0.1 | 0.1 | 0.4 | 0.1 | 0.1 | |
| Stearyl alcohol | 0.5 | 0.5 | 1.0 | 0.5 | 0.5 | 0.2 | 0.4 | 0.5 | | 0.7 | 0.5 |
| Stearic acid | 0.1 | | 0.1 | | 0.1 | 0.2 | 0.5 | 0.1 | 0.8 | 0.1 | 0.1 |
| Cetyl alcohol | 0.7 | 0.5 | 0.8 | 1.0 | 0.7 | 1.0 | 0.5 | | 0.7 | 0.7 | 1.0 |
| Behenyl Alcohol | | 0.5 | | | 0.5 | | | | | | |
| PEG 100 Stearate | 0.1 | 0.1 | | 0.3 | 0.4 | 0.5 | 0.1 | 0.1 | 0.1 | 0.1 | 0.1 |
| Decyl glucoside | | | | 0.2 | | | | | | | |
| Potassium | 0.1 | | | 0.1 | | 0.2 | | | 0.05 | | |
| Phosphate | | | | | | | | | | | |
| Tris | | 0.5 | | | | | 0.3 | | | | |
| ADA | | | 0.3 | | | | | 0.2 | | | 0.1 |
| Protease 1 | 0.1 | 0.1 | 0.01 | 0.001 | | | | 0.025 | | 0.05 | |
| Protease 2 | | | | | 0.05 | | 0.01 | | 0.01 | | 0.01 |
| Protease 3 | | | | | | 0.1 | 0.02 | | | | |
| Isohexadecane | 2.0 | 2.5 | 1.0 | 2.5 | | 3.0 | 2.0 | | 2.0 | 2.2 | 1.0 |
| Isopropyl | 0.7 | 0.5 | 0.7 | 1.0 | | 0.7 | 1.0 | | 1.5 | 0.5 | 0.1 |
| isostearate | | | | | | | | | | | |
| Sucrose | 0.3 | 0.3 | 0.3 | 0.1 | | 0.2 | 1.0 | | 0.5 | 0.5 | 0.5 |
| polycottonseedate | | | | | | | | | | | |
| Dimethicone/ | 1.0 | 2.0 | 1.0 | 2.5 | | 1.0 | 1.5 | 2.0 | 1.5 | 1.0 | 1.0 |
| dimethiconol | | | | | | | | | | | |
| Capric/caprylic | | | | | 2.0 | | | 1.0 | | | |
| triglyceride | | | | | | | | | | | |
| Petrolatum | | 2.0 | | | 2.0 | | | 2.0 | | | |
| Cyclomethicone/ | | | | | | | | 3.0 | | | 2.0 |
| dimethicone | | | | | | | | | | | |
| Colpolyol | | | | | | | | | | | |
| EDTA | 0.15 | | 0.15 | | 0.2 | 0.2 | | 0.14 | | 0.15 | |
| PH | 7.0 | 6.5 | 6.8 | 7.3 | 7.0 | 8.0 | 8.5 | 7.9 | 6.5 | 7.3 | 7.0 |

| | |
|---|---|
| Water | Q.S. to 100 |

Examples XII to XV may be formulated as gel/hydrogel patch and/or as face masks:

| | XII | XIII | XIV | XV |
|---|---|---|---|---|
| Inhibitor 1 | 0.5 | | | |
| Inhibitor 2 | | 0.2 | 0.5 | |
| Inhibitor 3 | | 0.1 | | 0.1 |
| Bicine | | | 8 | 5 |
| Potassium glycolate | | | | 1.5 |
| Calcium chloride dihydrate | 0.01 | 0.01 | 0.01 | |
| Formyl phenyl boronic acid | | | | 0.1 |
| Glycerin | | 5 | | 12 |
| Polyethylene glycol 200 | | | 2 | |
| Hexylene glycol | | 5 | | 2 |
| Polyvinylalcohol (31,000 mwt) | 5 | | 3 | 10 |
| Polyvinylalcohol (205,000 mwt) | 5 | | 8 | 6 |
| Agarose | | 2.0 | 1.0 | 0.5 |
| Xanthan/Locust bean | | 0.5 | 1.0 | 0.75 |
| Niacinamide | 8 | 5 | | |
| Panthenol | 1.0 | 1.0 | | 2.0 |
| Tocopherol nicotinate | | | 5.0 | |
| Decyl glucoside | 0.4 | | | |
| Potassium Phosphate | 0.1 | — | | |
| Tris | | | | 0.1 |
| ADA | | | 0.1 | |
| Protease 1 | 0.1 | 0.1 | | 0.01 |
| Protease 2 | | | 0.05 | |
| Protease 3 | 0.1 | | | 0.25 |
| EDTA | 0.1 | — | 0.1 | 0.1 |
| pH | 6.0 | 7.5 | 6.0 | 8.0 |

| | |
|---|---|
| Water | Q.S. to 100 |

Examples XXIII to XXVI may be formulated as water-in-oil emulsions:

| | XVI | XVII | XVIII | XIX |
|---|---|---|---|---|
| Inhibitor 1 | 0.5 | | | |
| Inhibitor 2 | | 0.2 | 0.1 | |
| Inhibitor 3 | | 1 | | 0.5 |
| Trimethyl glycine | | | 10 | |
| Sodium Formate | | | 1 | |
| Formyl phenyl boronic acid | 1 | | | 0.5 |
| Calcium chloride dihydrate | 0.01 | | 0.01 | 0.01 |
| Glycerin | 10 | 5 | | 5 |
| Polyethylene glycol 200 | | 5 | 15 | |
| 12% Dimethicone/vinyl dimethicone | 10 | 8 | | 20 |
| crosspolymer | | | | |
| 25% Dimethicone/copolyol cross polymer | 2.5 | 2.5 | | 2.5 |
| in dimethicone | | | | |
| Cyclomethicone/dimethicone copolyol | | | 10 | |
| Cyclomethicone | 5 | 2 | 20 | 5 |
| Decyl glucoside | | | 0.1 | |
| Niacinamide | 2 | 2 | 5 | |
| Panthenol | 1 | 1 | | |
| Tocopherol nicotinate | | | 1 | 0.1 |
| Retinyl propionate | | 0.25 | | |
| Tocopherol acetate | 0.5 | | | 0.5 |
| Polydimethylsilsequioxane | 1 | 1 | | 1 |
| Titanium dioxide | 0.5 | | | 0.5 |
| Polysorbate 40 | | | 2 | |
| Polyglyceryl-3 Diisostearate | | 0.5 | | |
| PPG-15 Stearyl ether | 1 | 1 | | |
| C12-15 Alkyl benzoate | 1 | | | |
| Potassium Phosphate | 0.2 | | | 0.1 |
| ADA | | 0.1 | 0.1 | |
| Protease 1 | 0.1 | | 0.01 | |
| Protease 2 | | 0.1 | | |
| Protease 3 | | | | 0.001 |
| Isopropyl palmitate | 1.0 | | | |
| Petrolatum | | 2.0 | | |
| EDTA | 0.15 | | | 0.15 |
| PH | 7.0 | 7.5 | 6.5 | 6.5 |

| | |
|---|---|
| Water | Q.S. |

What is claimed is:

1. A leave-on composition for topical application in personal care, comprising (a) a stabilized enzyme, (b) at least about 30%, by weight of the composition, of water, (c) less than about 5%, by weight of the composition, of an earth alkali metal salt, and (d) an enzyme inhibitor having an inhibition constant (Kᵢ) from about 10 nM to about 25,000 nM.

2. A composition according to claim 1 comprising:
(a) from about 0.0001 % to about 1%, by weight of the composition, of said enzyme;
(b) at least about 40%, by weight of the composition, of water;
(c) less than about 0.1%, by weight of the composition, of said earth alkali metal salt; and
(d) from about 0.00005% to about 5%, by weight of the composition, of said inhibitor.

3. A composition according to claim 2 comprising:
(a) from about 0.001 % to 0.1 %, by weight of the composition, of said enzyme;
(b) at least about 50%, by weight of the composition, of water;
(c) less than about 0.02%, by weight of the composition, of said earth alkali metal salt; and
(d) from about 0.005% to 2%, by weight of the composition, of said inhibitor.

4. A composition according to claim 1 wherein the molar ratio of enzyme to inhibitor is from about 2:1 to about 1:20.

5. A composition according to claim 4 wherein the molar ratio of enzyme to inhibitor is from about 1:1 to about 1:15.

6. A composition according to claim 1 wherein said enzyme is a protease.

7. A composition according to claim 6 wherein said protease is selected from the group consisting of subtilisin BPN′, subtilisin Carlsberg, subtilisin DY, subtilisin 147, subtilisin 168, subtilisin 309, and subtilisin amylosaccaritus; more preferably subtilisin BPN′ and variants thereof.

8. A composition according to claim 7 wherein said protease is a variant having an amino acid substitution at one or more of the following positions: 9, 31, 76, 77, 79, 122, 156, 169, 188, 206, 212, 217, 218, 222, 254, and 271.

9. A composition according to claim 8 wherein said variant is a subtilisin variant having one or more of the following amino acid substitutions: S9A, I31 L, N76D, N77D, I79A, I79E, I122A, E156S, G169A, S188P, Q206D, Q206L, Q206V, Q206C, N212G, Y217K, Y217L, N218S, M222A, M222Q, T254A, and Y271K.

10. A composition according to claim 9 wherein said subtilisin variant comprises a combination of substitutions selected from the group consisting of: (a) N76D-I122A-Y217L, (b) I79A-I122A-Y217L, and (c) N76D-I79A-I122A-Y217L.

11. A composition according to claim 10 wherein said subtilisin variant comprises the combination of amino acid substitutions N76D-I122A-Y217L.

12. A composition according to claim 1 wherein said inhibitor has a Kᵢ ranging from about 50 nM to about 5,000 nM.

13. A composition according to claim 12 wherein said inhibitor has a Kᵢ ranging from about 100 nM to about 1,000 nM.

14. A composition according to claim 1 wherein said inhibitor is selected from the group consisting of:

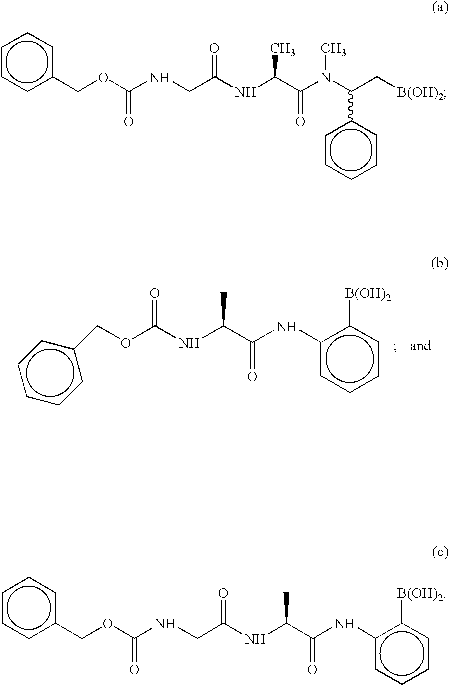

15. A composition according to claim 1 wherein said inhibitor is an enzyme inhibitor conjugate having the formula: 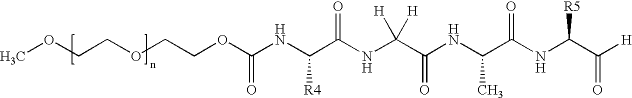
wherein R⁴ and R⁵ are each independently selected from the group consisting of substituted or unsubstituted: phenyl, benzyl, naphthyl, linear or branched C₁-C₇ alkyl, and mixtures thereof, and n is from 3 to 200.

16. A composition according to claim 15 wherein said enzyme inhibitor conjugate has the structure:

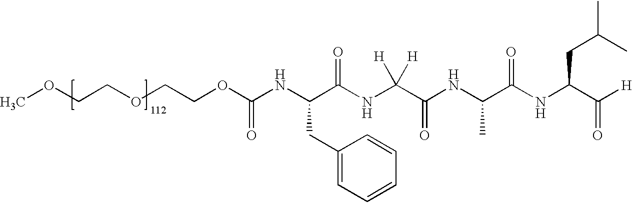

17. composition according to claim 1 wherein said inhibitor is selected from the group consisting of Streptomyces subtilisin inhibitor, those inhibitors having at least about 70% amino acid sequence homology to SSI, and variants of both.

18. composition according to claim 17 wherein said inhibitor is selected from the group consisting of variants of Streptomyces subtilisin inhibitor and those inhibitors having at least about 70% amino acid sequence homology to SSI, wherein said variants comprise one or more of the following substitutions: A62K, L63I, M73P, D83C, S98D, S98E.

19. A composition according to claim 1 further comprising from about 0.1% to about 50%, by weight of the composition, of a polyhydric alcohol.

20. A composition according to claim 19 comprising from about 1% to about 15%, of said polyhydric alcohol.

21. A composition according to claim 19 wherein said polyhydric alcohol is selected from the group consisting of glycerine, polyethylene glycol, hexane triol, butane-1,4-diol, butoxytriol, erythritol, xylitol, and mixtures thereof.

22. A composition according to claim 22 wherein said polyhydric alcohol is selected from the group consisting of xylitol, glycerine and polyethylene glycols having an average molecular weight of about 200 to about 400.

23. A composition according to claim 19 comprising from about 2% to about 20%, by weight of the composition, of glycerine.

24. A composition according to claim 19 wherein the ratio of polyhydric alcohol to water is from about 1:3 to about 1:50.

25. A composition according to claim 24 wherein the ratio of polyhydric alcohol to water is from about 1:5 to about 1:25.

26. A composition according to claim 19 wherein said polyhydric alcohol has a number average molecular weight of less than about 35,000.

27. A composition according to claim 26 wherein said polyhydric alcohol has a number average molecular weight of less than about 2,000.

28. A composition according to claim 1 further comprising from about 1% to about 50%, by weight of the composition, of an an osmo-protectant selected from the group consisting of:
(i) an osmo-protectant conforming to formula (I): 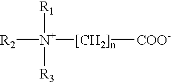
wherein R₁, R₂, and R₃ are independently selected from —H, —CH₃, —CH₂CH₃, and [—CH₂CH(OH)R₄, wherein R₄ is selected from —H, and C1 to C4 alkanes]; and
wherein n=an integer from 1 to 3;
(ii) an osmo-protectant conforming to formula (I), wherein any two of R₁, R₂, and R₃ are independently selected from —H, and —CH₃, and the third moiety of R₁, R₂, and R₃ is selected from —H and —(CH₂)mCH₃ wherein m is 4 or 5; and wherein n=an integer from 1 to 3;
(iii) an osmo-protectant conforming to formula (II): 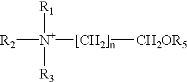
wherein R₁, R₂, R₃ R₄, and n are defined as in part (i) above; and wherein R₅ is selected from PO₃ and SO₃;
(iv) an osmo-protectant selected from the group consisting of alanine, glycine, serine, proline, carnitine, taurine, trimethylamineoxide, ectoin, and hydroxyectoin;
(v) an osmo-protectant selected from the group consisting of tricine, dimethyl proline, gamma-butyro betaine, beta-alanine betaine, valine betaine, lysine betaine, omithine betaine, alanine betaine, glutamic acid betaine, and phenyalanine betaine; and
(vi) mixtures thereof.

29. A composition according to claim 28 wherein:
(i) the osmo-protectant of (i) is selected from those osmo-protectants conforming to formula (I), wherein (R₁, R₂, and R₃ are all —CH₃, and n is 1), wherein (R₁, R₂, and R₃ are all —CH₃, and n is 3), wherein (R₁and R₂ are —CH₃, R₃ is —H, and n is 1), wherein (R₁ is —CH₃, R₂ and R₃ are —H, and n is 1), or wherein (R₁ and R₂ are —CH₂(OH)R₄, R₄ is —H, R₃ is —H, and n is 1);
(ii) the osmo-protectant of (ii) conforms to formula (I), wherein (R₁ and R₂ are —CH₃, R₃ is —(CH₂)mCH₃ wherein m is 5, and n is 1);
(iii) the osmo-protectant of (iii) conforms to formula (II), wherein (R₁, R₂, and R₃ are —CH₃; n is 1; and R₅ is PO₃);
(iv) the osmo-protectant of (iv) is selected from the group consisting of serine, proline, and carnitine; and
(vi) the osmo-protectant of (v) is selected from the group consisting of tricine and gamma-butyro betaine.

30. A composition according to claim 1 further comprising from about 0.1% to about 5%, by weight of the composition, of a lyotropic stabilizer.

31. A composition according to claim 30 wherein said lyotropic stabilizer is selected from the group consisting of salts of formate, glycolate, adipate, malonate, sulfate, phosphate, succinate, and mixtures thereof.

32. A composition according to claim 31 wherein the cation comprising said salt is selected from the group consisting of alkaline metals, ammonium, tris[hydroxymethyl]aminomethane, acetamide monoethanolamine, and triethanolamine.

33. A composition according to claim 32 wherein the cation comprising said salt is selected from the group consisting of sodium, potassium, lithium, and acetamide monoethanolamine.

34. A composition according to claim 1 further comprising an additional enzyme inhibitor selected from the group consisting of aryl boronic acid derivatives according to any of the following structures:

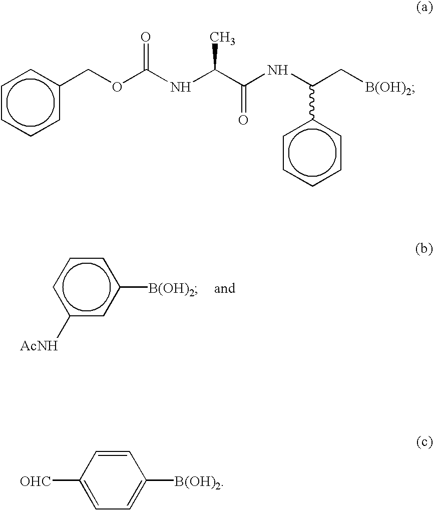

35. A composition according to claim 1 further comprising an additional enzyme inhibitor selected from the group consisting of boric acid, butylboronic acid, phenylboronic acid, leupeptide, and mixtures thereof.

36. A composition according to claim 1 further comprising a thickening agent selected from the group consisting of an ammonium acrylates/acrylamide crosspolymer having an acrylic acid to acrylamide ratio from 4:1 to 1000:1; polyacrylamide/AMPS copolymer; and xanthan gum.

37. A composition according to claim 1 further comprising from about 3% to about 25%, by weight of the composition, of a surfactant.

38. A composition according to claim 37 wherein said surfactant is selected from the group consisting of alkylpolyglucoses, alkyl ether sulfates, alkyl sulfates, soaps, monoalkylphosphates, acylsarcosinates, acylamphoacetates, alkyl amidopropylbetaines, alkylbetaines, alpha-olefin-sulfonates, alkylethoxylates, alkylpolyglycinates, alkylglycerolsulfonates, alkylmonoethanolamides, alkylethercarboxylates, acyl isethionates, akyl-modified dimethicone copolyols, acylglutamates, copolymers of polyethylene oxide/polypropylene oxide, and mixtures thereof.

39. A composition according to claim 38 wherein said surfactant is selected from the group consisting of decyl glucoside, lauryl glucoside, potassium lauryl phosphate, sodium lauryl sulfate, sodium laureth sulfate, ammonium lauryl sulfate, ammonium laureth sulfate sodium laurylamphoacetate, and mixtures thereof.

40. A composition according to claim 39 comprising from about 0.01% to about 2%, of decyl glucoside.

41. A composition according to claim 1 further comprising a skin-active agent selected from the group consisting of niacinamide, panthenol, farnesol, glucosamine, retinyl propionate, vitamin E, tocopherol acetate, tocopherol nicotinate, retinol, retinyl palmitate, retinoic acid, vitamin C, vitamin D, caffeine, theobromine, allantoin, alpha-bisabol, phytantriol, magnesium ascorbyl phosphate, ascorbyl glucoside, pyridoxine, palmityl penta-peptide-3, pitera, mevastatin, lovastatin, and mixtures thereof.

42. A composition according to claim 41 wherein said skin-active agent is selected from the group consisting of niacinamide, panthenol, farnesol, retinyl propionate, vitamin E, tocopherol acetate, tocopherol nicotinate, retinol, retinyl palmitate, retinoic acid, caffeine, theobromine, allantoin, alpha-bisabol, pyridoxine, palmityl penta-peptide-3, pitera, and mixtures thereof.

43. A composition according to claim 42 wherein said skin-active agent is formed as a complex comprising niacinamide, panthenol and a tocopherol acetate compound.

44. A composition according to claim 1 wherein said composition has a water activity greater than about 0.65.

45. A composition according to claim 44 wherein said composition has a water activity greater than about 0.85.

46. A composition according to claim 1 having an osmotic pressure of less than 10 atmospheres.

47. A leave-on composition for topical application in personal care, comprising (a) from about 0.001% to about 0.1%, by weight of the composition, of a subtilisin protease variant, (b) at least about 30%, by weight of the composition, of water, (c) less than about 0.05%, by weight of the composition, of an earth alkali metal salt, and (d) from about 0.005% to about 2%, by weight of the composition, of an enzyme inhibitor selected from the group consisting of (i) an enzyme inhibitor conjugate having the structure: 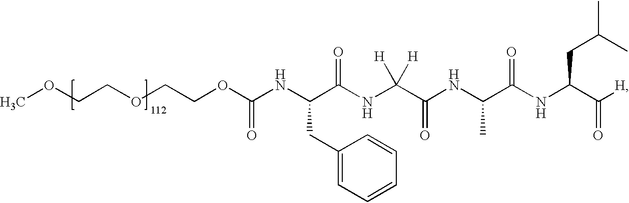
(ii) variants of SSI and SSI-like inhibitors having at least about 70% amino acid sequence homology with SSI, wherein the variants comprise one or more of the following substitutions: A62K, L63I, M73P, D83C, S98D, S98E, and (iii) polyethylene glycol-modified forms of SSI and SSI-like inhibitors having at least about 70% amino acid sequence homology.

48. A composition according to claim 1, wherein the composition is formulated as a cream, gel, hydrogel, gel patch, hydrogel patch, facemask, lotion, leave-on rinse, tonic, spray, ointment, poultice, foam, mousse, pomade, or paste.

49. A kit comprising (a) a composition according to claim 48 and (b) a set of instructions instructing a user to wipe off the composition after a period of time instead of washing said composition off.

50. A method of providing a skin care benefit selected from the group consisting of skin moisturization, skin softness, skin smoothness, and combinations thereof, comprising topically applying a safe and effective amount of a composition according to claim 1, to skin in need of such skin care benefit.

51. A method of providing a consumer with a leave-on enzyme-containing personal care product, wherein the enzyme is inhibited and inactive on shelf, but is active on skin without the need for a separate re-activation step, said leave-on enzyme-containing personal care product comprising a composition according to claim 1.
